(19) United States
(12) Patent Application Publication
(10) Pub. No.: US 2016/0193362 A1
(43) Pub. Date: Jul. 7, 2016

(54) NEW CELL-SPECIFICALLY ACTIVE NUCLEOTIDE MOLECULES AND APPLICATION KIT FOR THE APPLICATION THEREOF

(71) Applicant: Friedrich-Schiller-Universitaet Jena, Jena (DE)

(72) Inventors: Tobias POEHLMANN, Zwickau (DE); Rolf GUENTHER, Hamburg (DE)

(73) Assignee: Friedrich-Schiller-Universitaet Jena, Jena (DE)

(21) Appl. No.: 14/442,655

(22) PCT Filed: Nov. 14, 2013
(86) PCT No.: PCT/EP2013/073887

§ 371 Date: May 13, 2015

(30) Foreign Application Priority Data
Nov. 15, 2012 (DE) 10 2012 022 596.2

(51) Int. Cl. A61K 48/00; C12N 15/113; C12N 15/11

(52) U.S. Cl. CPC A61K 48/0041; C12N 15/11; C12N 15/113; C12N 2310/141; C12N 2310/3231; C12N 2310/14; C12N 2310/531; C12N 2310/3181; C12N 2310/3513; C12N 2320/32

(57) ABSTRACT

Cell-specifically active nucleotide molecules and application kit for the use thereof.

The problem to be solved was to modify long molecules in such a way that, by chemical modifications, their biological function is reliably inactivated and can be completely reactivated in a cell-specific manner.

According to the invention several peptides or polymers are bound to nucleotide molecules in such a way that theft spatial structure is modified to to such a degree that their biological function is no longer ensured or that molecules which normally anneal to the nucleic acids can no longer access the nucleic acids.

Said molecules are used in particular for cell-specifically influencing cells by introduction of nucleic acids.

### BACKGROUND OF THE INVENTION

The invention relates to new nucleotide-based biologically active molecules by means of which the expression of genes can be induced or reduced in a targeted manner in specific cells, and an application kit for use.

By introducing nucleic acids into cells, it is possible to achieve the production of genes or gene segments, proteins or protein fragments, which are encoded on the inserted DNA sequences, as well as of shorter or longer peptides; or, in the case of the insertion of interfering RNA molecules, the expression of a specific gene segment which is complementary to the RNA can be suppressed. Inhibition of the expression of genes can be achieved, amongst others, by insertion of siRNA (short interfering RNA) or miRNA (microRNA). Typically, upon activation, siRNA molecules can interact with the mRNA of the target gene and, in combination with specific endoribonucleases, they form an RNA protein complex designated “RISC” (RNA induced silencing complex). The RISC complex binds to the target mRNA wherein endonucleases cut the target mRNA. In this way, gene expression is prevented and, thus, the formation of target proteins is inhibited.

The inhibition of gene expression by the introduction of short (19-23 bp) double-stranded RNA molecules (siRNA) in eukaryotic cells which are specific for a sequence segment of the mRNA of the target gene has already been described (Elbashir S M et al.: Duplexes of 21-nucleotide RNAs mediate RNA interference in cultured mammalian cells, Nature, 2001, May 24, 411(6836), 494-8; Liu Y et al.: Efficient and isoform-selective inhibition of cellular gene expression by peptide nucleic acids, Biochemistry, 2004 Feb. 24, 43(7), 1921-7; U.S. Pat. No. 5,898,031 A; U.S. Pat. No. 7,056,704 B2).

The use of such molecules does not prevent the transcription of a gene and the production of an mRNA, but the siRNA initiates a cell mechanism which degrades the target mRNA. Finally, as described above, the generation of a specific protein is suppressed without interfering with the expression of further genes (post-transcriptional gene silencing).

At present, the use of siRNA often aims at suppressing exclusively the expression of one single gene in a cell. Thus, effects which silence several genes at the same time or in an unspecific manner are undesirable and, for this reason, the sequences of the mRNA are designed in such a way that these effects are suppressed.

Methods aiming at increasingly transfecting cells of a target tissue in vivo with siRNA (Ikeda et al.: “Ligand-Targeted Delivery of Therapeutic siRNA”, Pharmaceutical Research, Vol. 23, No. 8, August 2006) or at achieving cell specificity by the binding of short peptides which are cleaved in a cell-specific manner (WO 2008/098569 A2) were also developed. By using said modified siRNA molecules, it is possible to selectively reduce or suppress the expression of genes in specific cells.

These methods for the targeted effect of nucleic acids, however, are often limited to short nucleic acid sequences; with longer sequences, there is the problem that the molecules are instable and, thus, cannot be inserted into cells efficiently by means of targeted delivery; the known binding of short peptides at the ends of longer nucleic acids and their cell-specific cleavage often does not lead to the desired cell-specific effect, since the binding of peptides to the end of a long RNA or DNA sequence does not lead to a sufficient inactivation.

The problem underlying the invention is the modification of long nucleic acid molecules in such a way that their biological function is reliably inactivated by means of chemical modifications and, also, can be completely re-activated cell-specifically.

### SUMMARY OF THE INVENTION

According to the invention, several peptides or polymers are bound to nucleic acid molecules in such a way that their spatial structure is modified so drastically that their biological function is no longer guaranteed or that molecules which usually anneal to the nucleic acids can no longer access the nucleic acids.

The problem of the present invention is solved by single- or double-stranded nucleotide molecules which are of a length of more than 21 bases for insertion into cells which are characterised in that the nucleotide molecules, for their inactivation, are bound to at least one peptide or polymer which inhibits the biological activity of the molecules and which can be cleaved by enzymes and, thus, the biological activity can be re-activated. In one embodiment, for inhibition of the nucleotide molecules, at least one peptide or polymer can be bound between the ends of the nucleotides. In an alternative embodiment, for inhibition of the nucleotide molecules, at least one peptide or polymer can be bound to the backbone of the nucleotides in such a way that both ends are bound to one another. In a further embodiment also constructs are provided wherein for the inhibition of the nucleotide molecules, at least one peptide is bound between the ends of the nucleotides and, in addition, at least one peptide or polymer is bound to the backbone of the nucleotides in such a way that both ends are bound to one another.

Within the meaning of the above, the essence of the present invention is particularly based on long nucleic acid molecules being designed in such a way that their biological function is reliably inactivated by means of chemical modifications and also, can be completely re-activated cell-specifically. In the context of the present invention, the term “long nucleotide molecules” or “long nucleic acid molecules” does not only comprise molecules which are of a length of more than 21 bases. In particular, also nucleotide molecules or nucleic acid molecules are comprised which are of a length of more than 23 bases. Nucleotide molecules or nucleic acid molecules are preferred which are of a length of more than 25 bases. In a further preferred embodiment, long nucleic acid molecules within the meaning of the invention are modified by means of chemical modifications in such a way that their biological function is reliably inactivated and, also, can be completely re-activated cell-specifically, wherein the nucleic acid molecules or nucleotide molecules are of a length of more than 30, 40, 50 or more bases.

According to the invention, however, also single- or double-stranded nucleotide molecules are provided for the insertion into cells, which are characterised in that the nucleotide molecules, for their inactivation, are bound to at least one peptide or polymer which inhibits the biological activity of the molecules and which can be cleaved by enzymes and, thus, the biological activity is re-activated, with particularly molecules being provided which are of a length in the range of 23 to 10,000 bases.

Nucleotide molecules or nucleic acid molecules of the invention having a length within a range of 23, 25, 30, 40 or 50 to 100 bases, in particular within the range of 23 to 100 bases, are particularly preferred. Typically, these lengths are found in nucleotide molecules or nucleic acid molecules from the group of shRNAs, miRNAs and antisense-nucleotides, however, they are not limited thereto.

In a further preferred embodiment also single- or double-stranded nucleotide molecules are provided for insertion into cells which are characterised in that the nucleotide molecules, for their inactivation, are bound to at least one peptide or polymer which inhibits the biological activity of the molecules and which can be cleaved by enzymes and, thus, the biological activity is re-activated, with particularly molecules being provided which preferably are of a length in the range of 100 to 2000 bases. Typically, these lengths are found in nucleotide molecules or nucleic acid molecules from the group of synthetic mRNAs, Spiegelmers and aptamers, however, they are not limited thereto.

In a further preferred embodiment, also single- or double-stranded nucleotide molecules are provided for insertion into cells which are characterised in that the nucleotide molecules, for their inactivation, are bound to at least one peptide or polymer which inhibits the biological activity of the molecules and which can be cleaved by enzymes and, thus, the biological activity is re-activated, with particularly molecules being provided which preferably are of a length in the range of 2000 to 10000 bases. Typically, these lengths are found in nucleotide molecules or nucleic acid molecules such as mRNAs, however, they are not limited thereto.

Contrary to described methods, wherein short nucleotide molecules are biologically inactivated by binding of peptides or polymers, which is due to the structure of the peptides, in the present invention, it is suggested to design the peptides or polymers in such a way that the structure of the nucleotides is modified and, thus, their biological activity is inhibited. If peptides or polymers are cleaved from nucleotides by specific enzymes, they return to their original structure and develop their normal biological activity.

The cleavage by specific enzymes can particularly be induced in that with specific disease or development conditions of cells (in particular cell cycle or differentiation in stem cells), the enzymes exhibit an activity which is specific for specific cell types or disease-relevant modifications thereof (in particular degeneration or infection) or genotype-specific activity. Furthermore, specific cleavage can take place for the detection of specific enzymes or with the uses mentioned.

In this context, specific enzymes can be proteases or peptidases (caspases, amino peptidases or serine proteases; in particular caspase-1, caspase-2, caspase-3, caspase-4, caspase-5, caspase-6, caspase-7, caspase-8, KLK4, PLAP, IRAP, uPA, FAP-α or viral proteases, for example HIV proteases, Coxsackievirus protease, Epstein-Barr virus protease, hepatitis A, B, C virus protease), nucleases, glycosidases, saccharases or chitinases.

Due to the binding of peptides or polymers to e.g. micro (mi)RNA as described, it is possible to achieve the modification of 3D structures, which normally occur, by sequence homologies. When peptides or polymeres are bound to mRNA, it is possible to achieve that the initiation sites for the annealing to the mRNA are covered for translation and that, thus, the protein encoded on the mRNA is not expressed.

The selection of peptide or polymer bonds is not restricted to the ends of the nucleotide molecules, but binding may also occur at the sugar molecules of nucleotides, at the phosphates or the organic bases.

As already mentioned above, the nature of the single- or double-stranded nucleotide molecules of the present invention is not limited to specific nucleic acid molecule species or nucleotide molecule species. Thus, the single- or double-stranded nucleotide molecule of the invention can be an mRNA, an shRNA or a PNA. However, the single- or double-stranded nucleotide molecule of the invention can also be an aptamer or a Spiegelmer. In another embodiment, the single- or double-stranded molecules of the invention may be immunostimulating RNAs. Furthermore, the single- or double-stranded nucleotide molecule is not only provided in the form of one of the above-mentioned individual nucleotide molecule species. Rather, in a preferred embodiment, mixtures or mixed forms of the individual species (mRNA, DNA, shRNA, PNA, immuostimulating RNA, aptamer and/or Spiegelmer) of the single- or double-stranded nucleotide molecules of the invention are provided.

The term “aptamer” comprises short single-stranded DNA or RNA oligonucleotides which are capable of binding a specific molecule via their three-dimensional structure. The term “Spiegelmer” comprises L-ribonucleic acid aptamers (short L-RNA aptamers). L-ribonucleic acid aptamers are molecules similar to ribonucleic acid (RNA) which consist of L-ribonucleotides which do not occur naturally. They are artificial oligonucleotides and stereochemical mirrors of natural oligonucleotides. Thus, L-ribonucleic acid aptamers are a specific form of aptamers and, like these, they are capable of binding specific molecules via their three-dimensional structure. L-ribonucleic acid aptamers are known under their trade name “Spiegelmer”.

Immunostimulating RNAs within the meaning of the invention are RNA molecules which are capable of interacting with cell-specific molecule complexes, for example RIG-I (RIG-I (“retinoic acid-inducible gene I”) is a RIG-I-like receptor dsRNA helicase enzyme which is a member of the family of RIG-I-like receptors (RLR)). Through the interaction, a signal transduction cascade is activated and/or an immunoreaction and/or apoptosis is induced (for a review see Kawai and Akira, Ann N Y Acad Sci 1143:1-20 (2008) and Schlee et al., Immunol. Rev. 227(1):66-74 (2009)). These immunostimulating RNAs can be further characterized by a 5′ triphosphate.

It is advantageous to have an application kit for use and administration of the biologically inactive nucleotide molecules that consists at least of
at least one ampoule (ampoule A) which contains the biologically inactive nucleotide molecules and may further contain:
at least one further ampoule (ampoule B) with a transfection system, for example cell-penetrating peptides, nanoparticles, polyethylenimines or lipids,
dilution and reaction buffers for the contents of ampoules A and B
one or more probes and syringes with cannulas and other required materials for injecting the mixture from the ampoule contents into the medium containing the target cells as well as
instructions for use and administration.

In the following, the invention is to be illustrated in detail by embodiments shown in the drawings.

### BRIEF DESCRIPTION OF THE DRAWINGS

FIG. 1: an exemplary and schematic representation of an mRNA (1a) to which biological molecules or molecule complexes, for example ribosomes (2), anneal in the known manner and thereby induce a biological process. Furthermore, the modification of the mRNA (1b) by e. g. a peptide (3a) is shown, whereby the annealing of biological molecules or of molecule complexes, for example ribosomes (2), and thus the induction of a biological process is prevented. If the peptide or the peptides (3a) are again cleaved from the mRNA, the biological molecules or the molecule complexes can again anneal to the mRNA (1a) in the known manner and induce the known biological processes.

FIG. 2: an exemplary and schematic representation of an mRNA (1a) to which biological molecules or molecule complexes, for example ribosomes (2), anneal in the known manner and thereby induce a biological process. Furthermore, the modification of the mRNA (1b) by e. g. a peptide (3b), is shown, whereby the spatial structure of the mRNA is modified in such a way that the annealing of the biological molecules or of the molecule complexes, for example ribosomes (2), and thus the induction of a biological process are prevented. Said process can be enhanced by the formation of double strands of the RNA via random or correspondingly designed homologies. If the peptide or the peptides (3b) are again cleaved partially or completely from the mRNA, the spatial structure of the mRNA is modified once more and the biological molecules or molecule complexes (2) can again anneal to the mRNA (1a) in the known manner and induce the known biological processes.

### DETAILED DESCRIPTION OF THE INVENTION

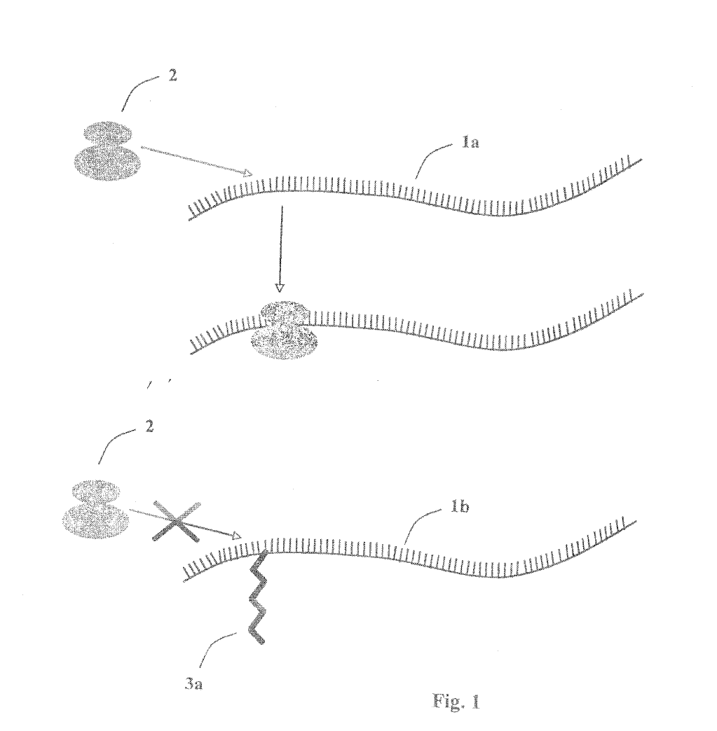

FIG. 1 shows the exemplary mechanism by means of an mRNA (1a). Normally, for example ribosomes (2) anneal to the mRNA and thereby induce a biological process, in the case of ribosomes translation. By the modification of the exemplary mRNA (1b) by a bound exemplary peptide (3a), the annealing of, for example, the ribosomes (2) is prevented. For this reason, in the case of ribosomes (2), no translation of the mRNA (1b) can occur. If the peptide (3a) is cleaved, for example by an enzyme, the annealing of the exemplary ribosome (2) to the mRNA is no longer prevented and the normal biological process, in the case of ribosomes translation, takes place. The binding of the peptide (3a) can occur at the initiation site of the exemplary ribosomes or at another site of the mRNA; depending on the binding site, either the annealing of the ribosomes (2) or the complete transcription of the exemplary mRNA is prevented. In either case, the normal biological function of the mRNA can no longer be fulfilled upon binding of the exemplary peptide.

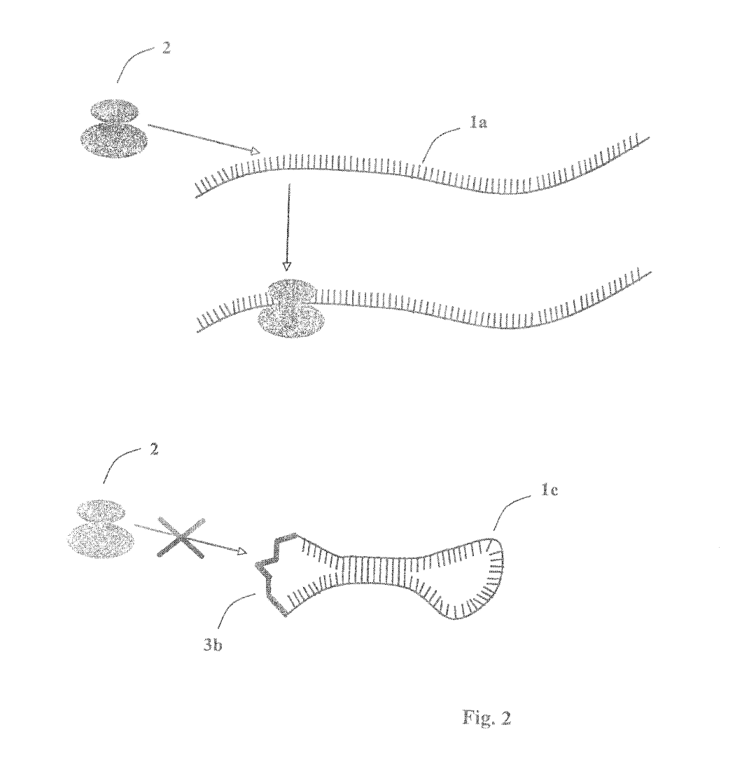

FIG. 2 shows a further possible exemplary mechanism by means of an mRNA (1a). In this case, normally for example ribosomes (2) anneal to the mRNA and induce a biological process, in the case of ribosomes, translation. By the modification of the exemplary mRNA (1c) by a bound exemplary peptide (3b), the spatial structure of the exemplary mRNA is modified in such a manner that the annealing of, for example, ribosomes (2) is prevented. For this reason, in the case of ribosomes (2), no translation of the mRNA (1c) can occur. If for example the peptide (3c) is cleaved, for example by an enzyme, the annealing of the exemplary ribosome (2) to the mRNA (1a) is no longer prevented and the normal biological process, in the case of ribosomes translation, takes place.

Examples of use:
1) Induction of the production of toxic proteins in target cells: the RNA can be selected in such a way that its sequence encodes one or several segments of a toxic protein or peptide or that it encodes a complete toxic protein or peptide. Examples are bacterial toxins such as for example diphtheria toxin, anthrax A toxin, anthrax B toxin, botulinum toxin or toxins of higher organisms (conidae, snakes, lizards, insects, spiders, scorpions).
2) Induction of the production of allergens in target cells, in particular in combination with transport sequences which ensure the display of allergens on the cell surface so that they become accessible to the immune system. It is particularly preferred to combine the allergen with an HLA sequence, in particular in sequential order or with the allergen at a site within the HLA sequence so that allergen and HLA sequence are presented together on the cell surface. Particularly suitable allergens are non-human allergens such as ambrosia.
3) Specific induction of the production of HLA proteins which are presented on the cell surface, for example for the induction of immune tolerance after transplantation of tissues or organs (transplantation medicine).

1. Inactivated single or double-stranded nucleotide molecules having biological activity before being inactivated and having a length of more than 21 bases and being in an inactivated state on account of being bound to a substance selected from the group consisting of at least one peptide or polymer which inhibits the biological activity of said molecules and which can be cleaved by enzymes whereby the biological activity is re-activated.

2. The inactivated nucleotide molecules of claim 1, wherein the substance is at least one peptide containing a cleaving sequence of proteases.

3. The inactivated nucleotide molecules of claim 2, wherein the at least one peptide is bound to a backbone of the nucleotide molecules with both ends of the nucleotides bound to one another.

4. The inactivated nucleotide molecules of claim 2, wherein the at least one peptide is bound between ends of the nucleotides.

5. The inactivated nucleotide molecules of claim 1, wherein the substance is at least one polymer which can be cleaved by enzymes.

6. The inactivated nucleotide molecules of claim 1, wherein the substance is a combination of at least one polymer and at least one peptide which can be cleaved by enzymes.

7. The inactivated nucleotide molecules of claim 1, wherein the nucleotide molecule is an mRNA.

8. The inactivated nucleotide molecules of claim 1, wherein the nucleotide molecule is an miRNA.

9. The inactivated nucleotide molecules of claim 1, wherein the nucleotide molecule is a DNA.

10. The inactivated nucleotide molecules of claim 1, wherein the nucleotide molecule is an shRNA.

11. The inactivated nucleotide molecules of claim 1, wherein the nucleotide molecule is a PNA.

12. The inactivated nucleotide molecules of claim 1, wherein the nucleotide molecules contain LNAs.

13. Application kit for use and administration of nucleotide molecules according to claim 1, comprising
at least one ampoule (ampoule A) which contains the biologically inactivated nucleotide molecules;
at least one further ampoule (ampoule B) which contains a transfection system;
dilution and reaction buffers for the contents of ampoules A and B;
one or more probes and syringes with cannulas and other required materials for injecting a mixture of the ampoule contents into a medium containing target cells; and
instructions for use and administration.

14. The application kit of claim 13, wherein the transfection system comprises cell-penetrating peptides, nanoparticles,
